(19) United States
(12) Patent Application Publication
(10) Pub. No.: US 2005/0059047 A1
(43) Pub. Date: Mar. 17, 2005

(54) Plant SUGI homologs

(76) Inventors: Joan Tellefsen Odell, Unionville, PA (US); Stephen M. Allen, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/871,620

(22) Filed: Jun. 18, 2004

Related U.S. Application Data
(63) Continuation of application No. 09/462,972, filed on Jan. 14, 2000, now abandoned.

(51) Int. Cl. C12Q 1/68; C07H 21/04; C12N 9/10; C12N 9/88; A01H 1/00; C12N 15/82

(52) U.S. Cl. 435/6; 435/69.1; 435/193; 435/419; 435/468; 536/23.2; 800/284

(57) ABSTRACT

This invention relates to an isolated nucleic acid fragment encoding a plant SUG1 protein, a homolog of the mouse SUG1 protein. The invention also relates to the construction of a chimeric gene encoding all or a portion of the plant SUG1 protein, in sense or antisense orientation, wherein expression of the chimeric gene results in production of altered levels of the plant SUG1 protein in a transformed host cell.

### FIELD OF THE INVENTION

This invention is in the field of plant molecular biology. More specifically, this invention pertains to nucleic acid fragments encoding coactivator proteins involved in regulation of gene expression in plants and seeds. These same proteins also function as regulatory components of the 26S proteasome which is involved in regulation of protein turnover.

### BACKGROUND OF THE INVENTION

Gene expression levels are influenced by the interactions of transcription factors with proteins that are present in general transcription complexes. In addition, other non-DNA binding proteins known as coactivators interact with transcription factors and transcription complex proteins to further stimulate transcription. These coactivators are thought to be mediators of the transcription response. One type of coactivator is represented by the SUG1 gene, which has been identified in several types of eucaryotic cells such as mouse, yeast and Xenopus cells. SUG1 proteins have a coiled coil domain and an “AAA” domain (i.e., ATPases associated with a variety of cellular activities; see vom Bauer et al. (1996) EMBO J. 15:110-124).

SUG1 proteins have been shown to bind to the activation domains of the transcription factors, for example yeast SUG1 has been shown to bind to GAL4 of yeast and VP16 of herpesvirus (Swaffield et al. (1995) Nature 374:88-91). A mutant GAL4 that lacks the activation domain and therefore cannot activate transcription recovers this function in the presence of a mutant yeast SUG1, indicating a coactivation role for SUG1 (Swaffield et al. (1992) Nature 357:698-700). Yeast SUG1 has also been shown to associate with the TATA-binding protein of the TFIID transcription complex in vivo and in vitro, indicating an intermediary role in gene activation (Swaffield et al. (1995) Nature 374:88-91).

Mouse SUG1 has been shown to be able to complement a yeast SUG1 mutant, suggesting that the mouse SUG1 also functions as a transcription coactivator (vom Bauer et al. supra). The human thyroid receptor interacting protein 1 (Trip1), which differs from the mouse SUG1 protein by only three amino acids, also complements a yeast SUG1 mutant and binds to the Gal4 and VP16 activation domains (Lee et al. (1995) Nature 374:91-94). Interestingly, Trip1 also binds to the thyroid-hormone receptor and the retinoid-X receptor in ligand-dependent fashion which further suggests that Trip1 plays a role in mediating transcription.

The mouse SUG1 protein may also play a role in protein degradation. Amino acid comparisons indicate that the mouse SUG1 sequence differs by only a single amino acid residue from the human p45 protein, which was isolated as a subunit of the PA700 proteasome regulatory complex (vom Bauer et al. (1996) EMBO J 15:110-124). PA700 includes a set of regulatory proteins that are associated with the 26S proteasome, a complex that carries out selective degradation of abnormal proteins and naturally short-lived proteins related to cell cycle control and metabolic regulation (Akiyama et al. (1995) FEBS Letters 363:151-156).

To date no SUG1 protein homologs have been reported in plants. Because these proteins are involved in transcriptional regulation they are obvious targets for manipulating gene regulation in eukaryotes and as possible targets for screening assays for crop protection chemicals. There is a great deal of interest in identifying the genes that encode SUG1 homologs in plants. These genes may be used to express SUG1 proteins in plant cells to manipulate gene expression. Accordingly, the availability of nucleic acid sequences encoding all or a portion of a SUG1 protein would facilitate studies to better understand and alter gene expression in plants.

### SUMMARY OF THE INVENTION

The instant invention relates to an isolated nucleic acid fragment encoding a plant coactivator involved in regulation of gene expression. Moreover, the protein encoded by the isolated nucleic acid fragment also regulates protein turnover. More particularly, this invention concerns isolated nucleic acid fragments encoding plant (specifically from maize, soybean, rice and wheat) homologs of the mouse SUG1 transcriptional coactivator. In addition, this invention relates to a nucleic acid fragment that is complementary to a nucleic acid fragment encoding a plant SUG1 protein.

In another embodiment, the instant invention relates to a chimeric gene that comprises a nucleic acid fragment encoding a plant SUG1 protein, or to a chimeric gene that comprises a nucleic acid fragment that is complementary to a nucleic acid fragment encoding a plant SUG1 protein, the nucleic acid fragment operably linked to suitable regulatory sequences, wherein expression of the chimeric gene results in production of levels of the encoded protein in transformed host cells that are altered (i.e., increased or decreased) relative to the levels produced in untransformed host cells.

In a further embodiment, the instant invention concerns a transformed host cell comprising in its genome a chimeric gene comprising a nucleic acid fragment encoding a plant SUG1 protein or a chimeric gene comprising a nucleic acid fragment that is complementary to the nucleic acid fragment encoding a plant plant SUG1 protein, the chimeric gene operably linked to suitable regulatory sequences. Expression of the chimeric gene results in production of altered levels of protein encoded by the operably linked nucleic acid fragment in the transformed host cell. The transformed host cell can be of eukaryotic or prokaryotic origin, and include cells derived from higher plants and microorganisms. The invention also includes transformed plants that arise from transformed host cells of higher plants, and seeds derived from such transformed plants.

An additional embodiment of the instant invention concerns a method of altering the level of expression of a plant SUG1 protein in a transformed host cell comprising: a) transforming a host cell with a chimeric gene comprising a nucleic acid fragment encoding a plant SUG1 protein or a chimeric gene that comprises a nucleic acid fragment that is complementary to the nucleic acid fragment encoding a plant SUG1 protein; and b) growing the transformed host cell under conditions that are suitable for expression of the chimeric gene wherein expression of the chimeric gene results in production of altered levels of the protein encoded by the operably linked nucleic acid fragment in the transformed host cell.

An addition embodiment of the instant invention concerns a method for obtaining a nucleic acid fragment encoding all or substantially all of an amino acid sequence encoding a plant SUG1 protein.

A further embodiment of the instant invention is a method for evaluating at least one compound for its ability to inhibit the activity of a plant SUG1 protein, the method comprising the steps of: (a) transforming a host cell with a chimeric gene comprising a nucleic acid fragment encoding a plant SUG1 protein operably linked to suitable regulatory sequences; (b) growing the transformed host cell under conditions that are suitable for expression of the chimeric gene wherein expression of the chimeric gene results in production of the plant SUG1 protein in the transformed host cell; (c) optionally purifying the plant SUG1 protein expressed by the transformed host cell; (d) treating the plant SUG1 protein with a compound to be tested; and (e) comparing the activity of the plant SUG1 protein that has been treated with a test compound to the activity of an untreated plant SUG1 protein, thereby selecting compounds with potential for inhibitory activity.

### BRIEF DESCRIPTION OF THE DRAWINGS AND SEQUENCE DESCRIPTIONS

The invention can be more fully understood from the following detailed description and the accompanying drawings and sequence descriptions which form a part of this application.

FIG. 1 shows a comparison of the amino acid sequences of the mouse SUG1 protein (Z54219), the human p45 protein (P47210), the human Trip1 transcriptional coactivator (L38810), the Saccharomyces cerevisiae SUG1 transcriptional coactivator (X66400), and the instant soybean and maize SUG1 protein homologs (se1.pk0023.b5 and cs1.pk0051.b7, respectively).

FIG. 2 shows a comparison of the amino acid sequences of the mouse SUG1 protein SEQ ID NO:7 (3), the instant soybean clone se1.pk0023.b5 (2), a corn contig (cs1.pk0051.b7, cr1n.pk0096.b2 and cta1n.pk0056.c11) (1) and a wheat clone (w11n.pk0053.g3) (4).

The following sequence descriptions and sequence listings attached hereto comply with the rules governing nucleotide and/or amino acid sequence disclosures in patent applications as set forth in 37 C.F.R. §1.821-1.825.

SEQ ID NO:1 is the nucleotide sequence comprising the entire cDNA insert in clone se1.pk0023.b5 encoding a soybean SUG1 protein.

SEQ ID NO:2 is the deduced amino acid sequence of a soybean SUG1 homolog derived from the nucleotide sequence of SEQ ID NO:1.

SEQ ID NO:3 is the nucleotide sequence comprising the entire cDNA insert in clone cs1.pk0051.b7 encoding corn SUG1 protein.

SEQ ID NO:4 is the deduced amino acid sequence of a portion of a maize SUG1 homolog derived from the nucleotide sequence of SEQ ID NO:3.

SEQ ID NO:5 is the nucleotide sequence comprising a contig assembled from the cDNA inserts in clones cs1.pk0051.b7, cr1n.pk0096.b2 and cta1n.pk0056.c11 encoding a corn SUG1 protein.

SEQ ID NO:6 is the deduced amino acid sequence of a SUG1 protein derived from the nucleotide sequence of SEQ ID NO:5.

SEQ ID NO:7 is the amino acid sequence encoding the mouse SUG1 protein having EMBL accession No. Z54219.

SEQ ID NO:8 is the amino acid sequence encoding the Saccharomyces cerevisiae SUG1 transcriptional coactivator having EMBL accession No. X66400.

SEQ ID NO:9 is the amino acid sequence encoding the human Trip1 transcriptional coactivator having GenBank accession No. L38810.

SEQ ID NO:10 is the amino acid sequence encoding the human p45 protein having SWISS-PROT accession No. P47210.

SEQ ID NO:11 is the nucleotide sequence comprising a portion of a cDNA insert in clone w11n.pk0053.g3 encoding a wheat SUG1.

SEQ ID NO:12 is the deduced amino acid sequence of a wheat SUG1 protein from the nucleotide sequence of SEQ ID NO:11.

SEQ ID NO:13 is the nucleotide sequence comprising a portion of a cDNA insert in clone r1r6.pk0064.e10 encoding a rice 26S protease regulatory subunit 8.

SEQ ID NO:14 is the deduced amino acid sequence of a 26S protease regulatory subunit 8 protein from the nucleotide sequence of SEQ ID NO:13.

The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

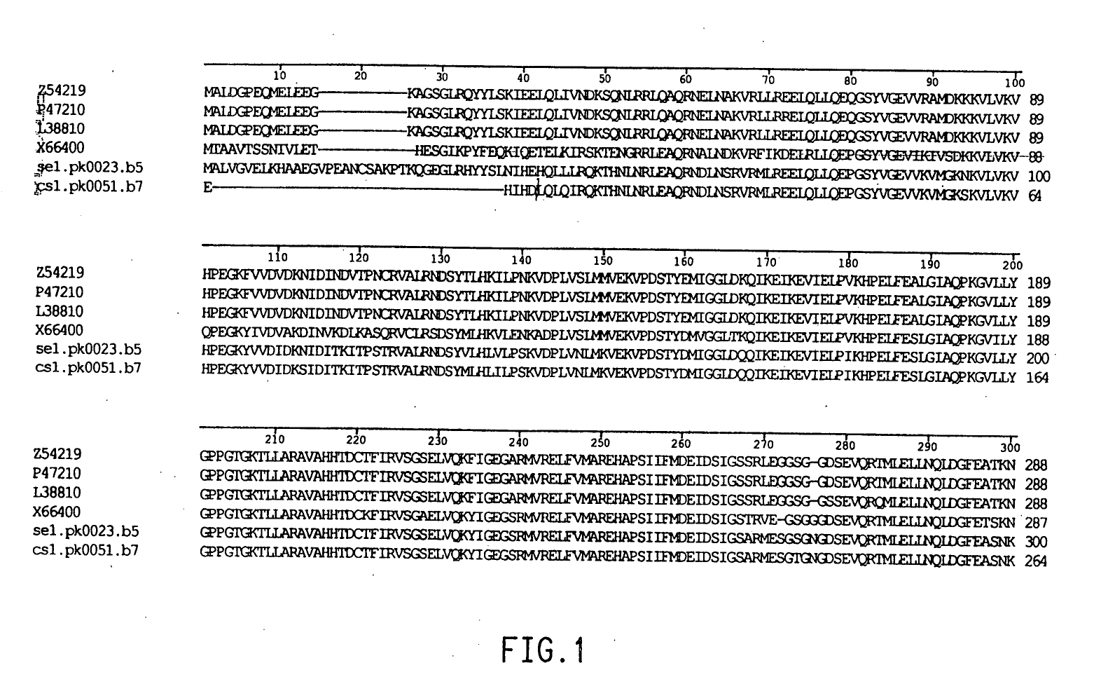

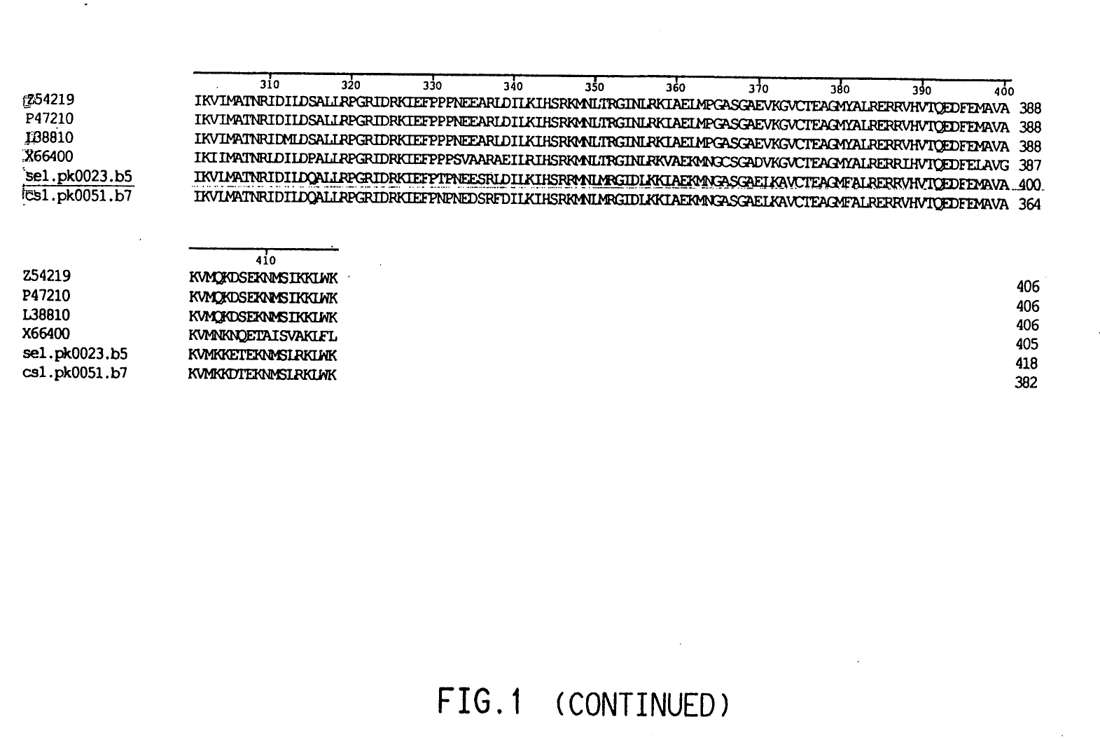

The amino acid sequence similarity between the instant plant SUG1 proteins and the mouse and yeast SUG1 proteins, human Trip1 and p45 proteins indicates that the plant SUG1 proteins may function as transcriptional coactivators and as regulators of protein degredation (FIG. 1). The plant proteins may therefore be used to reduce expression of specific genes whose promoters are normally regulated by SUG1, using antisense or co-suppression technology. The instant plant proteins may also be used to enhance gene expression of those genes whose promoters are normally targeted by the transcription factors that the plant SUG1 proteins normally interact with.

Alternatively, the plant SUG1 protein coactivation function can be targeted to a novel promoter region by the addition of either a DNA binding domain or a protein-protein interaction domain. The instant plant SUG1 protein can be fused to a very defined DNA-binding domain, such as, but not limited to, a bacterial lexA DNA binding domain, a yeast Gal4 DNA-binding domain or a DNA binding domain from a plant transcription factor. On the other hand, a synthetic promoter can be designed to contain multiple copies of a target site which is necessary for the specific binding by either the lexA, Gal4 or plant DNA binding domain. By using this approach, the plant SUG1 protein can be specifically targeted to the engineered synthetic promoter, thus leading to a higher level of gene expression when used in combination with an interacting transcription factor.

Additionally, plant SUG1 proteins can be fused to a transcription factor that already includes its own DNA binding domain in order to target the coactivator. Besides DNA-binding domains, the plant SUG1 proteins can also be fused to other transcription regulatory proteins, such as transcription mediators. Normally, these mediators do not bind to DNA directly and are recruited to their target sites by interaction with other DNA-binding proteins. By fusing the plant SUG1 protein to these mediators, the plant SUG1 protein can be targeted to specific regulatory elements through the interaction between the mediators and other DNA-binding proteins.

In the context of this disclosure, a number of terms shall be utilized. As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. As used herein, “contig” refers to an assemblage of overlapping nucleic acid sequences to form one contiguous nucleotide sequence. For example, several DNA sequences can be compared and aligned to identify common or overlapping regions. The individual sequences can then be assembled into a single contiguous nucleotide sequence.

As used herein, “substantially similar” refers to nucleic acid fragments wherein changes in one or more nucleotide bases results in substitution of one or more amino acids, but do not affect the functional properties of the protein encoded by the DNA sequence. “Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate alteration of gene expression by antisense or co-suppression technology or alteration of the functional properties of the resulting protein molecule. It is therefore understood that the invention encompasses more than the specific exemplary sequences.

For example, it is well known in the art that antisense suppression and co-suppression of gene expression may be accomplished using nucleic acid fragments representing less than the entire coding region of a gene, and by nucleic acid fragments that do not share 100% sequence identity with the gene to be suppressed. Moreover, alterations in a gene which result in the production of a chemically equivalent amino acid at a given site, but do not effect the functional properties of the encoded protein, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products. Moreover, the skilled artisan recognizes that substantially similar nucleic acid sequences encompassed by this invention are also defined by their ability to hybridize, under stringent conditions (0.1×SSC, 0.1% SDS, 65° C.), with the sequences exemplified herein. Preferred substantially similar nucleic acid fragments of the instant invention are those nucleic acid fragments whose DNA sequences are 85% identical to the coding sequence of the nucleic acid fragments reported herein. More preferred nucleic acid fragments are 90% identical to the coding sequence of the nucleic acid fragments reported herein. Most preferred are nucleic acid fragments that are 95% identical to the coding sequence of the nucleic acid fragments reported herein. The percent identity used herein, can be precisely determined by the DNASTAR protein alignment protocol using the Hein algorithm (Hein, J. J. (1990) Unified Approach to Alignment and Phylogenies. Methods in Enzymology, vol. 183, 626-645). Default parameters for the J. J. Hein method for multiple alignments are: GAP PENALTY=11, GAP LENGTH PENALTY=3; for pairwise alignments KTUPLE 6.

A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to afford specific identification and/or isolation of a nucleic acid fragment comprising the sequence. The instant specification teaches partial or complete amino acid and nucleotide sequences encoding a particular plant protein. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment that encodes all or a substantial portion of the amino acid sequence encoding a corn, soybean, rice or wheat SUG1 protein as set forth in SEQ ID NOs:2, 4, 6, 12 and 14. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host Determination of preferred codons can be based on a survey of genes derived from the host cell where sequence information is available.

“Gene” refers to a nucleic acid fragment that encodes a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a DNA sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of different lengths may have identical promoter activity.

The translation leader sequence“refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript. Alternatively, the RNA transcript may be an RNA sequence derived from posttranscriptional processing of the primary transcript; this is referred to as the mature RNA. “Messenger RNA” (mRNA) refers to the RNA that is without introns and that can be translated into protein by the cell. “cDNA” refers to a double-stranded DNA that is complementary to and derived from mRNA. “Sense” RNA refers to an RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that is not translated yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020).

“Altered levels” refers to the production of gene product(s) in transgenic organisms in amounts or proportions that differ from that of normal or non-transformed organisms.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Maniatis”).

This invention relates to plant cDNAs with homology to the mouse, yeast and Xenopus SUG1 proteins. The cDNAs have been isolated and identified by comparison of random plant cDNA sequences to several publically available databases using the BLAST algorithms well known to those skilled in the art. The nucleotide sequence of a soybean cDNA clone (se1.pk0023.b5) encoding a SUG1 homolog is provided in SEQ ID NO:1, and the deduced amino acid sequence is provided in SEQ ID NO:2. The nucleotide sequence of a maize SUG1 homolog (cs1.pk0051.b7) is provided in SEQ ID NOs:3 and 5, and the deduced amino acid sequence is provided in SEQ ID NO:4 and 6. The nucleotide sequence of a wheat SUG1 homolog (w11n.pk0053.g3) is provided in SEQ ID NO:11, and the deduced amino acid sequence is provided in SEQ ID NO:12. Lastly, the nucleotide sequence of a rice 26S protease regulatory subunit 8 homolog (r1r6.pk0064.e10) is provided in SEQ ID NO:13, and the deduced amino acid sequence is provided in SEQ ID NO:14. Homologs of these proteins from other plants can now be identified by comparison of random cDNA sequences to the maize, soybean, and wheat sequences provided herein.

The full insert of cDNA clone se1.pk0023.b5 encoding the soybean SUG1 homolog has been completely sequenced. Amino acid sequence comparison indicates that there is 80% sequence identity between this soybean homolog and the mouse SUG1 protein (FIG. 1). Sequence alignments and percent identity calculations were performed by the Jotun Hein method using the Megalign program of DNAStar™ sequence analysis software (DNASTAR Inc. 1228 South Park Street, Madison Wis., 53715)

Based on the high sequence homology, it is believed that this soybean clone encodes a plant homolog of mouse SUG1. This is the first indication that there is a similar SUG-mediated gene activation system in plants.

Likewise, the full insert of cDNA clone cs1.pk0051.b7 encoding the maize SUG1 homolog has been completely sequenced. The region encoding approximately thirty amino acids is missing from the 5′ end of the cDNA insert. At the amino acid level, this maize peptide is approximately 95% identical to the soybean SUG1 protein encoded by cDNA clone se1.pk0023.b5. Nucleotide identity between the soybean and maize cDNAs is approximately 78%. Sequence alignments and percent identity calculations were performed by the Jotun Hein method using the Megalign program of DNAStar™ sequence analysis software (DNASTAR Inc. 1228 South Park Street, Madison Wis., 53715).

The nucleic acid fragments of the instant invention may be used to isolate cDNAs and genes encoding other homologs of SUG1 from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other plant homologs of SUG1 either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full length cDNA or genomic fragments under conditions of appropriate stringency. Genomic fragments can be isolated that include the promoter region that directs expression of the plant SUG1 protein. This promoter may be prepared as a DNA fragment including regulatory elements with or without the untranslated leader and used in expression of other coding regions or for co-suppression.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al., (1988) PNAS USA 85:8998) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., (1989) PNAS USA 86:5673; Loh et al., (1989) Science 243:217). Products generated by the 3′ and 5′ RACE procedures can be combined to generate full-length cDNAs (Frohman, M. A. and Martin, G. R., (1989) Techniques 1:165).

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner, R. A. (1984) Adv. Immunol. 36:1; Maniatis).

The nucleic acid fragments of the instant invention may be used to create transgenic plants in which the disclosed plant homologs of SUG1 are present at higher or lower levels than normal or in cell types or developmental stages in which it is not normally found. This would have the effect of altering the level of a plant SUG1 protein in those cells.

Overexpression of plant a SUG1 protein may be accomplished by first constructing a chimeric gene in which the coding region is operably linked to a promoter capable of directing expression of a gene in the desired tissues at the desired stage of development. For reasons of convenience, the chimeric gene may comprise a promoter sequence and translation leader sequence derived from the same gene. A 3′ non-coding sequence encoding a transcription termination signal may also be provided. The instant chimeric gene may also comprise one or more introns in order to facilitate gene expression.

Plasmid vectors comprising the instant chimeric gene can then constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications, it may be desirable to reduce or eliminate expression of the genes encoding the instant plant of SUG1 homologs. In order to accomplish this, chimeric genes designed for co-suppression of the instant plant SUG1 protein genes can be constructed by linking the genes or gene fragments encoding the plant SUG1 proteins to plant promoter sequences. Alternatively, chimeric genes designed to express antisense RNA for all or part of the instant nucleic acid fragments can be constructed by linking the genes or gene fragments in reverse orientation to plant promoter sequences. Either the co-suppression or antisense chimeric genes could be introduced into plants via transformation wherein expression of the corresponding endogenous genes are reduced or eliminated.

The instant plant SUG1 homologs (or portions thereof) may be produced in heterologous host cells, particularly in the cells of microbial hosts, and can be used to prepare antibodies to the SUG1 proteins by methods well known to those skilled in the art. The antibodies are useful for detecting plant SUG1 proteins in situ in cells or in vitro in cell extracts. Preferred heterologous host cells for production of the instant SUG1 proteins are microbial hosts. Microbial expression systems and expression vectors containing regulatory sequences that direct high level expression of foreign proteins are well known to those skilled in the art. Any of these could be used to construct chimeric genes for production of the instant plant SUG1 homologs. These chimeric genes could then be introduced into appropriate microorganisms via transformation to provide high level expression of corn, soybean or wheat SUG1 proteins. An example of a vector for high level expression of the instant plant SUG1 homologs in a bacterial host is provided (Example 6).

Additionally, the instant SUG1 proteins can be used as targets to facilitate design and/or identification of inhibitors of the activity of the protein that may be useful as herbicides. This is desirable because the protein described plays a key role in regulation of gene expression. Accordingly, inhibition of the activity of the proteins described herein could lead to inhibition of gene expression sufficient to inhibit plant growth. Thus, the instant SUG1 proteins could be appropriate for new herbicide discovery and design.

All or a portion of the nucleic acid fragments of the instant invention may also be used as probes for genetically and physically mapping the genes that they are a part of, and as markers for traits linked to expression of the instant plant SUG1 proteins. Such information may be useful in plant breeding in order to develop lines with desired phenotypes.

For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et at., (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein, D. et al., (1980) Am. J. Hum. Genet.32:314-331).

The production and use of plant gene-derived probes for use in genetic mapping is described in R. Bernatzky, R and Tanksley, S. D. (1986) Plant Mol. Biol. Reporter 4(1):37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel, J. D., et al., In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask, B. J. (1991) Trends Genet. 7:149-154). Although current methods of FISH mapping favor use of large clones (several to several hundred KB; see Laan, M. et al. (1995) Genome Research 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian, H. H. (1989) J. Lab. Clin. Med. 114(2):95-96), polymorphism of PCR-amplified fragments (CAPS; Sheffield, V. C. et al. (1993) Genomics 16:325-332), allele-specific ligation (Landegren, U. et al. (1988) Science 241:1077-1080), nucleotide extension reactions (Sokolov, B. P. (1990) Nucleic Acid Res. 18:3671), Radiation Hybrid Mapping (Walter, M. A. et al. (1997) Nature Genetics 7:22-28) and Happy Mapping (Dear, P. H. and Cook, P. R. (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer, (1989) Proc. Natl. Acad. Sci USA 86:9402; Koes et al., (1995) Proc. Natl. Acad. Sci USA 92:8149; Bensen et al., (1995) Plant Cell 7:75). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant SUG1 gene. Alternatively, the SUG1 gene may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous SUG1 gene can be identified and obtained. This mutant plant can then be used to determine or confirm the natural functon of the plant SUG1 gene product.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Composition of cDNA Libraries: Isolation and Sequencing of cDNA Clones

cDNA libraries representing mRNAs from soybean embryo obtained seven days after flowering and maize leaf sheath obtained from five week old Zea mays B73 maize plants were prepaired. cDNA libraries representing mRNAs from wheat leaf from 7 day old etiolated seedlings (normalized, normalized essentially as described in U.S. Pat. No. 5,482,845) were prepaired. A cDNA librarie representing mRNAs from rice leaf 15 days after germination and 6 hours after infection of strain Magaporthe grisea 4360-R-62 (AVR2-YAMO) was also prepaired.

Separate cDNA libraries representing the maize, soybean, rice and wheat cDNAs were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences. Amplified insert DNAs were sequenced in dye-primer sequencing reactions to generate partial cDNA sequences (expressed sequence tags or “ESTs”; see Adams, M. D. et al., (1991) Science 252:1651). The resulting ESTs were analyzed using a Perkin Elmer Model 377 fluorescent sequencer.

### Example 2

### Identification and Characterization of cDNA Clones

ESTs encoding homologs of the mouse, yeast and Xenopus SUG1 proteins were identified by conducting BLAST (Basic Local Alignment Search Tool; Altschul, S. F., et al., (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

### Example 3

### Characterization of cDNA Clones Encoding SUG1 Homologs

The BLASTX search using the EST sequences from clones se1.pk0023.b5, cs1.pk0051.b7, cr1n.pk0096.b2 and cta1n.pk0056.c11 revealed similarity of the proteins encoded by the cDNAs to the SUG1 protein from mouse (EMBL Accession No. Z54219). The BLASTX search using the EST sequence from clone w11n.pk0053.g3 revealed similarity of the protein encoded by the cDNA to the SUG1 protein from Xenopus laevis (GenBank Accession No. X81986). The BLASTX search using the EST sequence from clone r1r6.pk0064.e10 revealed similarity of the protein encoded by the cDNA to the 26S Protease Regulatory Subunit 8 (a SUG1 protein homolog) from Dictyostelium discoideum (SWISS PROT Accession No. P34124). The BLAST results for each of these ESTs are shown in Table 3:

TABLE 1
BLAST Results for Clones Encoding Polypeptides Homologous
to SUG1 Proteins

| | BLAST pLog Score |
|---|---|
| Clone | (Organism Accession Number) |
| se1.pk0023.b5 | 35.05 (Z54219) |
| cs1.pk0051, b7 | 27.85 (Z54219) |
| Contig composed of: | 217.03 (Z54219) |
| cs1.pk0051.b7 | |
| ta1n.pk0056.c11 | |
| cr1n.pk0096.b2 | |
| wl1n.pk0053.g3 | 21.62 (X81986) |
| r1r6.pk0064.e10 | 53.19 (P34124) |

In the process of comparing the corn ESTs it was found that clones cs1.pk0051.b7, cr1n.pk0096.b2 and cta1n.pk0056.c11 had overlapping regions of homology. Using this homology it was possible to align the entire cDNA insert of clone cs1.pk0051.b7, with the ESTs of clones cr1n.pk0096.b2 and cta1n.pk0056.c11 and assemble a contig (a contig is an assemblage of overlapping nucleic acid sequences to form one contiguous nucleotide sequence). The individual sequences were assembled into a unique contiguous nucleotide sequence encoding a unique corn SUG1 protein. The nucleotide sequence of the contig is shown in SEQ ID NO:5; the deduced amino acid sequence of the contig is shown in SEQ ID NO:6. The nucleotide sequence of the cDNA clone w11n.pk0053.g3 is shown in SEQ ID NO:11; the deduced amino acid sequence encoded by clone w11n.pk0053.g3 is shown in SEQ ID NO:12. The nucleotide sequence of the cDNA clone r1r6.pk0064.e10 is shown in SEQ ID NO:13; the deduced amino acid sequence encoded by clone r1r6.pk0064.e10 is shown in SEQ ID NO: 14. BLAST scores and probabilities indicate that the instant nucleic acid fragments encode portions of a plant SUG1 protein. These sequences represent plant sequences encoding a SUG1 protein.

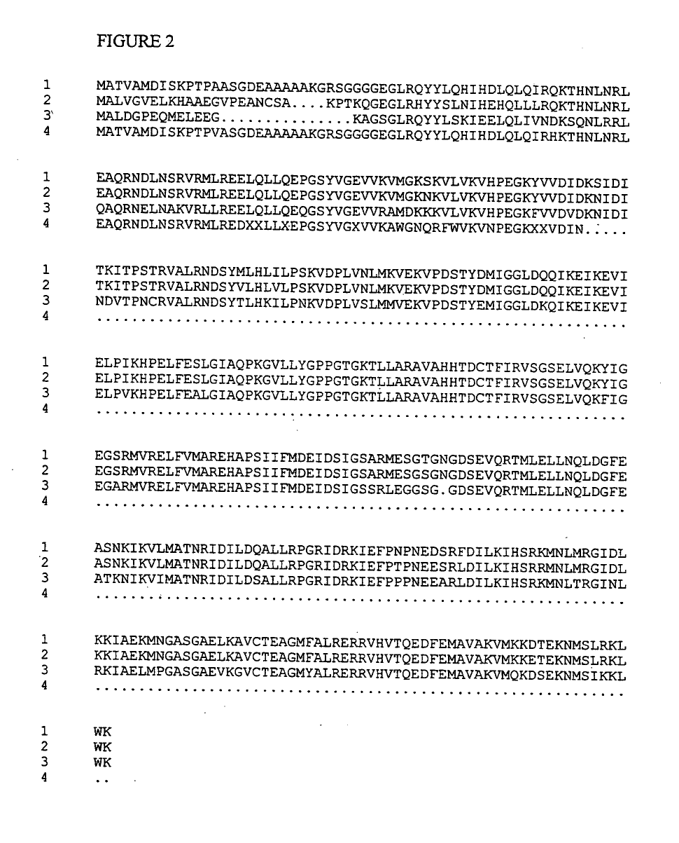

The sequence of the entire cDNA insert in clone se1.pk0023.b5 was determined and is shown in SEQ ID NO:1; the deduced amino acid sequence of this cDNA is shown in SEQ ID NO:2. The amino acid sequence set forth in SEQ ID NO:2 was evaluated by BLASTP, yielding a pLog value of 210.70 versus the mouse SUG1 sequence. The sequence of the entire maize contig containing clones cs1.pk0051.b7, cta1n.pk0056.c11 and cr1n.pk0096.b2 was determined and is shown in SEQ ID NO:5; the deduced amino acid sequence of this cDNA is shown in SEQ ID NO:6. The amino acid sequence set forth in SEQ ID NO:6 was evaluated by BLASTP, yielding a pLog value of 217.03 versus the mouse SUG1 sequence. FIG. 1 presents an alignment of the amino acid sequences set forth in SEQ ID NOs:2 the mouse SUG1 sequence. FIG. 2 presents an alighnment of the amino acid sequence set forth in SEQ ID NO:6 and the mouse SUG1 protein. The data in Table 2 represents a calculation of the percent identity of the amino acid sequences set forth in SEQ ID NOs:2 and 6 and the mouse SUG1 sequence.

TABLE 2
Percent Identity of Amino Acid Sequences Deduced From
the Nucleotide Sequences of cDNA Clones Encoding
Polypeptides Homologous to SUG1

| | | Percent Identity to |
|---|---|---|
| Clone | SEQ ID NO. | Z54219 |
| sel.pk0023.b5 | 2 | 80 |
| Contig composed of: | 6 | 81 |
| cs1.pk0051.b7 | | |
| cta1n.pk0056.c11 | | |
| cr1n.pk0096.b2 | | |

Sequence alignments and percent identity calculations were performed by the Jotun Hein method using the Megalign program of DNAStar™ sequence analysis software (DNASTAR Inc. 1228 South Park Street, Madison Wis., 53715).

Sequence alignments and BLAST scores and probabilities indicate that the instant nucleic acid fragments encode entire or nearly entire SUG1 protein. These sequences represent the first plant sequences encoding a SUG1 protein.

### Example 4

### Expression of Chimeric Genes in Monocot Cells

A chimeric gene comprising a cDNA encoding a plant SUG1 homolog in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Boulevard, Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding a plant SUG1 protein, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al., (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al., (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al., (1990) Bio/Technology 8:833-839).

### Example 5

### Expression of Chimeric Genes in Dicot Cells

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant plant SUG1 protein in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

A nucleic acid fragment encoding a plant SUG1 protein may be generated by polymerase chain reaction (PCR) of the instant cDNA clones using appropriate oligonucleotide primers. Cloning sites can be incorporated into the oligonucleotides to provide proper orientation of the DNA fragment when inserted into the expression vector. Amplification is then performed, and the isolated fragment is inserted into a pUC18 vector carrying the seed expression cassette.

Soybean embroys may then be transformed with the expression vector comprising sequences encoding a plant SUG1 protein. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Kline et al. (1987) Nature (London) 327:70, U.S. Pat. No. 4,945,050). A Du Pont Biolistic™ PDS1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the plant SUG1 protein, and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Expression of Chimeric Genes in Microbial Cells

For expression of a plant SUG1 protein, in a microbial cell, a cDNA encoding the instant plant SUG1 proteincan be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the SUG1 protein coding sequence. This fragment may then be purified on a 1% NuSieve GTG™ low melting agarose gel (FMC). Buffer and agarose contain 10 μg/ml ethidium bromide for visualization of the DNA fragment. The fragment can then be purified from the agarose gel by digestion with GELase™ (Epicentre Technologies) according to the manufacturer's insructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as decribed above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the plant SUG1 proteinare then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

### Example 7

### Evaluating Compounds for Their Ability to Inhibit Plant SUG1 Protein Activity

The plant SUG1 homologs described herein may be produced using any number of methods known to those skilled in the art. Such methods include, but are not limited to, expression in bacteria as described in Example 5, or expression in eukaryotic cell culture, in planta, and using viral expression systems in suitably infected organisms or cell lines. The instant polypeptide may be expressed either as a mature form of the protein as observed in vivo or as a fusion protein by covalent attachment to a variety of enzymes, proteins or affinity tags. Common fusion protein partners include glutathione S-transferase (“GST”), thioredoxin (“Trx”), maltose binding protein, and C— and/or N-terminal hexahistidine polypeptide (“(His)₆”). The fusion protein may be engineered with a protease recognition site at the fusion point so that fusion partner can be separated by protease digestion to yield an intact, mature polypeptide. Examples of such proteases include thrombin, enterokinase and factor Xa. However, any protease can be used which specifically cleaves the peptide connecting the fusion protein and the plant SUG1 polypeptide.

Purification of the instant polypeptide, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. When the polypeptide is expressed as a fusion protein, the purification protocol may include the use of an affinity resin which is specific for the fusion protein tag attached to the expressed polypeptide or an affinity resin containing ligands which are specific for the polypeptide. For example, polypeptide may be expressed as a fusion protein coupled to the C-terminus of thioredoxin. In addition, a (His)₆ peptide may be engineered into the N-terminus of the fused thioredoxin moiety to afford additional opportunities for affinity purification. Other suitable affinity resins could be synthesized by linking the appropriate ligands to any suitable resin such as Sepharose-4B. In an alternate embodiment, a thioredoxin fusion protein may be eluted using dithiothreitol; however, elution may be accomplished using other reagents which interact to displace the thioredoxin from the resin. These reagents include β-mercaptoethanol or other reduced thiol. The eluted fusion protein may be subjected to further purification by traditional means as stated above, if desired. Proteolytic cleavage of the thioredoxin fusion protein and the plant SUG1 protein may be accomplished after the fusion protein is purified or while the protein is still bound to the ThioBond™ affinity resin or other resin.

Crude, partially purified or purified polypeptide, either alone or as a fusion protein, may be utilized in assays for the evaluation of compounds for their ability to inhibit the activity of a plant SUG1 protein. Assays may be conducted under well known experimental conditions which permit optimal activity. An example of an in vitro assay for transcriptional coactivation activity is described by vom Bauer et al. ((1996) EMBO J. 15:110-124). The skilled artisan is well aware of simple modifications that could be made to the published protocols that would afford detection of inhibitors the plant SUG1 protein.

1. An isolated nucleic acid fragment encoding a plant SUG1 protein comprising a member selected from the group consisting of:
(a) an isolated nucleic acid fragment encoding all or a substantial portion of the amino acid sequence set forth in a member selected from the group consisting of SEQ ID NOs:2, 4, 6, 12, and 14;
(b) an isolated nucleic acid fragment that is substantially similar to an isolated nucleic acid fragment encoding all or a substantial portion of the amino acid sequence set forth in a member selected from the group consisting of SEQ ID NOs:2, 4, 6, 12, and 14; and
(c) an isolated nucleic acid fragment that is complementary to (a) or (b).

2. The isolated nucleic acid fragment of claim 1 wherein the nucleotide sequence of the fragment is set forth in a member selected from the group consisting of SEQ ID NOs:1, 3, 5,11, and 13.

3. A chimeric gene comprising the nucleic acid fragment of claim 1 operably linked to suitable regulatory sequences.

4. A transformed host cell comprising the chimeric gene of claim 3.

5. A method of altering the level of expression of a plant SUG1 protein in a host cell comprising:
(a) transforming a host cell with the chimeric gene of claim 3; and
(b) growing the transformed host cell produced in step (a) under conditions that are suitable for expression of the chimeric gene
wherein expression of the chimeric gene results in production of altered levels of a plant SUG1 protein in the transformed host cell.

6. A method of obtaining a nucleic acid fragment encoding all or substantially all of the amino acid sequence encoding a plant SUG1 protein comprising:
(a) probing a cDNA or genomic library with the nucleic acid fragment of claim 1;
(b) identifying a DNA clone that hybridizes with the nucleic acid fragment of claim 1;
(c) isolating the DNA clone identified in step (b); and
(d) sequencing the cDNA or genomic fragment that comprises the clone isolated in step (c)
wherein the sequenced nucleic acid fragment encodes all or substantially all of the amino acid sequence encoding a plant SUG1 protein.

7. A method of obtaining a nucleic acid fragment encoding a portion of an amino acid sequence encoding a plant SUG1 protein comprising:
(a) synthesizing an oligonucleotide primer corresponding to a portion of the sequence set forth in any of SEQ ID NOs:1, 3, 5, 11, and 13; and
(b) amplifying a cDNA insert present in a cloning vector using the oligonucleotide primer of step (a) and a primer representing sequences of the cloning vector
wherein the amplified nucleic acid fragment encodes a portion of an amino acid sequence encoding a plant SUG1 protein.

8. The product of the method of claim 6.

9. The product of the method of claim 7.

10. A method for evaluating at least one compound for its ability to inhibit the activity of a plant SUG1 protein, the method comprising the steps of:
(a) transforming a host cell with a chimeric gene comprising a nucleic acid fragment encoding a plant SUG1 protein, operably linked to suitable regulatory sequences;
(b) growing the transformed host cell under conditions that are suitable for expression of the chimeric gene wherein expression of the chimeric gene results in production of the SUG1 proteinen coded by the operably linked nucleic acid fragment in the transformed host cell;
(c) optionally purifying the SUG1 protein expressed by the transformed host cell;
(d) treating the SUG1 protein with a compound to be tested; and
(e) comparing the activity of the SUG1 protein that has been treated with a test compound to the activity of an untreated SUG1 protein,
thereby selecting compounds with potential for inhibitory activity.


SEQUENCE LISTING (14 sequences)

SEQ ID NO: 1 - DNA, 1254 bp, Glycine max
  CDS (1)..(1254)
atggctcttgtaggagttgaactgaagcatgcggcggagggcgtaccggaggcgaattgctccgccaagcccaccaagcagggcgagggcctccgccactactattctctcaacatccacgagcatcagctccttcttcgccaaaagactcataacctcaaccgtctcgaggctcagagaaacgacctcaattctagggtgaggatgctgcgcgaagaattacagcttctgcaggaacccggctcttatgtcggtgaagttgtcaaagtaatgggcaagaacaaagtccttgtcaaggtccacccagaaggaaaatatgttgttgacattgacaaaaatattgacattacaaagattactccatccactagagttgcactccgcaacgacagttatgttcttcacttagttctgccaagtaaagttgatccattggtcaatctgatgaaagttgagaaagttcccgattctacatatgacatgattggtggtttagaccagcaaattaaagaaataaaagaggtcattgagctaccaatcaaacatcctgagctgtttgaaagtcttggaattgcacaaccaaagggtgtcctgctctatgggccacctggtacaggtaaaacattgttggctagggcagtggctcatcatactgactgtacattcatcagggtgtctggttctgagttagttcagaaatacattggagaaggttctagaatggtcagggaactttttgttatggccagggaacatgctccatcaattatcttcatggatgaaattgacagtattggatctgctcggatggaatctggaagtggcaacggtgatagtgaggtacagcgtactatgctggaacttctcaaccagttggatggatttgaagcttcaaataagatcaaggttttgatggccaccaatcggattgatatcctggatcaagccctccttagaccaggacggatagaccggaaaattgaatttccaacccctaatgaagagtctcggctggatattttgaaaatccattctagaagaatgaatttaatgcgtggcattgatttgaagaagattgccgagaagatgaatggagcatctggtgctgaacttaaggctgtttgcactgaagctggaatgtttgctttgagggagcggagggtacacgtgactcaggaggattttgagatggccgtggcgaaggtgatgaaaaaggagactgaaaaaaacatgtcattgcggaagttgtggaag

SEQ ID NO: 2 - protein, 418 aa, Glycine max
MALVGVELKHAAEGVPEANCSAKPTKQGEGLRHYYSLNIHEHQLLLRQKTHNLNRLEAQRNDLNSRVRMLREELQLLQEPGSYVGEVVKVMGKNKVLVKVHPEGKYVVDIDKNIDITKITPSTRVALRNDSYVLHLVLPSKVDPLVNLMKVEKVPDSTYDMIGGLDQQIKEIKEVIELPIKHPELFESLGIAQPKGVLLYGPPGTGKTLLARAVAHHTDCTFIRVSGSELVQKYIGEGSRMVRELFVMAREHAPSIIFMDEIDSIGSARMESGSGNGDSEVQRTMLELLNQLDGFEASNKIKVLMATNRIDILDQALLRPGRIDRKIEFPTPNEESRLDILKIHSRRMNLMRGIDLKKIAEKMNGASGAELKAVCTEAGMFALRERRVHVTQEDFEMAVAKVMKKETEKNMSLRKLWK

SEQ ID NO: 3 - DNA, 1148 bp, Zea mays
  CDS (3)..(1148)
gagagcacatccatgacctgcagctccagatccggcagaagacccataacctcaaccgcctcgaggcccagcgcaacgacctcaactcccgagttagaatgctcagggaagagttgcagttgcttcaagagcctggctcatatgttggtgaggtggtgaaggtcatggggaaatcaaaggttctggtgaaggtacatcccgaaggcaaatatgtggtggatatagataagagcattgatatcactaagatcacaccttcaacaagagttgctcttcggaatgacagctatatgctccatctgatcctaccaagcaaagttgatccattggtcaatctcatgaaagttgagaaggttccggattctacctatgatatgattggaggccttgaccagcaaattaaagagatcaaagaggtcattgagcttccaatcaaacatccggaactgtttgagagccttggaattgcgcaaccaaagggtgtccttctttatggacctccgggcacaggaaagacattgttggcacgtgcggttgctcatcacactgactgcaccttcatcagggtgtctggttctgagttggttcagaagtatattggtgagggctcccggatggttagggaactctttgttatggccagggaacatgcaccatccattatatttatggatgaaattgactctatcggatctgctagaatggagtctggaactggcaacggtgacagtgaagttcagcgtactatgcttgaacttctaaaccagctcgatggttttgaagcatcaaacaaaattaaggttttgatggcaacgaacagaatagacattttggatcaagcccttctgaggcctggccgcatagacaggaagattgaatttccaaatcctaacgaggattcacgtttcgatatcttgaagatccattcaagaaaaatgaacttgatgcgtggcattgatctgaaaaagatcgcggaaaagatgaatggggcctcaggagctgagctcaaggccgtctgcacagaggctggaatgtttgctcttcgtgagagaagggtgcacgttacccaggaggacttcgagatggcagtggccaaggtgatgaagaaagacacggagaagaacatgtccctgcgcaagctctggaag

SEQ ID NO: 4 - protein, 382 aa, Zea mays
EHIHDLQLQIRQKTHNLNRLEAQRNDLNSRVRMLREELQLLQEPGSYVGEVVKVMGKSKVLVKVHPEGKYVVDIDKSIDITKITPSTRVALRNDSYMLHLILPSKVDPLVNLMKVEKVPDSTYDMIGGLDQQIKEIKEVIELPIKHPELFESLGIAQPKGVLLYGPPGTGKTLLARAVAHHTDCTFIRVSGSELVQKYIGEGSRMVRELFVMAREHAPSIIFMDEIDSIGSARMESGTGNGDSEVQRTMLELLNQLDGFEASNKIKVLMATNRIDILDQALLRPGRIDRKIEFPNPNEDSRFDILKIHSRKMNLMRGIDLKKIAEKMNGASGAELKAVCTEAGMFALRERRVHVTQEDFEMAVAKVMKKDTEKNMSLRKLWK

SEQ ID NO: 5 - DNA, 1358 bp, Zea mays
cccaagccaacaagcaagaccgcttctgcttgtgatccgaaacctttcttcgaacgcaaaaagaaccccacccgacgtaccgcgagccggcgatggcgacggtggcgatggacatctcgaagcccacgccggcagcgtccggtgacgaggcagcagcggcggcgaaggggaggagcggcggcgggggcgaggggctgcggcagtactacctgcagcacatccatgacctgcagctccagatccggcagaagacccataacctcaaccgcctcgaggcccagcgcaacgacctcaactcccgagttagaatgctcagggaagagttgcagttgcttcaagagcctggctcatatgttggtgaggtggtgaaggtcatggggaaatcaaaggttctggtgaaggtacatcccgaaggcaaatatgtggtggatatagataagagcattgatatcactaagatcacaccttcaacaagagttgctcttcggaatgacagctatatgctccatctgatcctaccaagcaaagttgatccattggtcaatctcatgaaagttgagaaggttccggattctacctatgatatgattggaggccttgaccagcaaattaaagagatcaaagaggtcattgagcttccaatcaaacatccggaactgtttgagagccttggaattgcgcaaccaaagggtgtccttctttatggacctccgggcacaggaaagacattgttggcacgtgcggttgctcatcacactgactgcaccttcatcagggtgtctggttctgagttggttcagaagtatattggtgagggctcccggatggttagggaactctttgttatggccagggaacatgcaccatccattatatttatggatgaaattgactctatcggatctgctagaatggagtctggaactggcaacggtgacagtgaagttcagcgtactatgcttgaacttctaaaccagctcgatggttttgaagcatcaaacaaaattaaggttttgatggcaacgaacagaatagacattttggatcaagcccttctgaggcctggccgcatagacaggaagattgaatttccaaatcctaacgaggattcacgtttcgatatcttgaagatccattcaagaaaaatgaacttgatgcgtggcattgatctgaaaaagatcgcggaaaagatgaatggggcctcaggagctgagctcaaggccgtctgcacagaggctggaatgtttgctcttcgtgagagaagggtgcacgttacccaggaggacttcgagatggcagtggccaaggtgatgaagaaagacacggagaagaacatgtccctgcgcaagctctggaag

SEQ ID NO: 6 - protein, 422 aa, Zea mays
MATVAMDISKPTPAASGDEAAAAAKGRSGGGGEGLRQYYLQHIHDLQLQIRQKTHNLNRLEAQRNDLNSRVRMLREELQLLQEPGSYVGEVVKVMGKSKVLVKVHPEGKYVVDIDKSIDITKITPSTRVALRNDSYMLHLILPSKVDPLVNLMKVEKVPDSTYDMIGGLDQQIKEIKEVIELPIKHPELFESLGIAQPKGVLLYGPPGTGKTLLARAVAHHTDCTFIRVSGSELVQKYIGEGSRMVRELFVMAREHAPSIIFMDEIDSIGSARMESGTGNGDSEVQRTMLELLNQLDGFEASNKIKVLMATNRIDILDQALLRPGRIDRKIEFPNPNEDSRFDILKIHSRKMNLMRGIDLKKIAEKMNGASGAELKAVCTEAGMFALRERRVHVTQEDFEMAVAKVMKKDTEKNMSLRKLWK

SEQ ID NO: 7 - protein, 406 aa, Mus musculus
MALDGPEQMELEEGKAGSGLRQYYLSKIEELQLIVNDKSQNLRRLQAQRNELNAKVRLLREELQLLQEQGSYVGEVVRAMDKKKVLVKVHPEGKFVVDVDKNIDINDVTPNCRVALRNDSYTLHKILPNKVDPLVSLMMVEKVPDSTYEMIGGLDKQIKEIKEVIELPVKHPELFEALGIAQPKGVLLYGPPGTGKTLLARAVAHHTDCTFIRVSGSELVQKFIGEGARMVRELFVMAREHAPSIIFMDEIDSIGSSRLEGGSGGDSEVQRTMLELLNQLDGFEATKNIKVIMATNRIDILDSALLRPGRIDRKIEFPPPNEEARLDILKIHSRKMNLTRGINLRKIAELMPGASGAEVKGVCTEAGMYALRERRVHVTQEDFEMAVAKVMQKDSEKNMSIKKLWK

SEQ ID NO: 8 - protein, 405 aa, Saccharomyces cerevisiae [sequence omitted]

SEQ ID NO: 9 - protein, 406 aa, Homo sapiens [sequence omitted]

SEQ ID NO: 10 - protein, 406 aa, Homo sapiens [sequence omitted]

SEQ ID NO: 11 - DNA, 548 bp, Triticum aestivum
  unsure (262): n = a, g, c or t
ccccgaccgacctaccctaatcggcgatggcgacggtggcgatggacatctcgaagcccactccagtggcgtccggcgacgaggccgcggcggcggccaaggggaggagcggcggagggggcgaggggctgcggcagtactacctgcagcacatccacgacctgcagctccagatccggcataagacccacaacctcaaccgtctcgaggcccagcgcaatgacctcaactcccgagttagaatgctcagggaagattgcanttgcttcangagcctggctcatatgttggtnaggtggtgaaggcatggggaaatcaaaggttctgggtnaaggtaaaccccgaaggcaaatannttgttgatataaataanccattgattcncangttnacacctncaacaanancgccttcgaantgatacnanngcccatctgttttaccaccaaattnncattggcaaccccatgaanttaaaagtcctgatcacataaaanctttggggcctgccacccttnagaaatanngttcttgtttcaanaaaacgg

SEQ ID NO: 12 - protein, 115 aa, Triticum aestivum
  UNSURE (78)..(79): Xaa = ANY AMINO ACID
MATVAMDISKPTPVASGDEAAAAAKGRSGGGGEGLRQYYLQHIHDLQLQIRHKTHNLNRLEAQRNDLNSRVRMLREDXXLLXEPGSYVGXVVKAWGNQRFWVKVNPEGKXXVDIN

SEQ ID NO: 13 - DNA, 593 bp, Oryza sativa
  unsure (149): n = a, c, g or t
gtttaaacggtacaggaaaaacgctactagctcgggcagttgctcatcacaccgactgtacctttattaagggtgtcaggttccgagttggttcagaagtatattggagagggttccagaatggttcgtgaactatttgtgatggccanagagcatgcaccatccataatctttatggatgaaatagactccattggatctgctanaatgcagtcangatctgggggtggtgatagtgaggttcaacgcactatgcttgatcttctgaatcaacttgatggctttgaagcatcaaacanaattaaggtcttatggcgacaaatangatggatattttggatcaagctctctgangcctggtcgcattgataggaagatgaatttccaatccgaatgaagatccgcttgatanttgaagatcattcaagaaaaatgactgatctggattgtctgaaaagatgcagagaaatgatgggcnctggacaaactaagngtctnacgaacaagatttgcctccaaanaggtattcacagagatctatgggtgcaaggtgaaagnacgaagaatccgncaccgaataaganncaccaacc

SEQ ID NO: 14 - protein, 126 aa, Oryza sativa
  UNSURE (20): Xaa = ANY AMINO ACID
GTGKTLLARAVAHHTDCTFXRVSGSELVQKYIGEGSRMVRELFVMAXEHAPSIIFMDEIDSIGSAXMQSXSGGGDSEVQRTMLDLLNQLDGFEASNXIKVXMATNXMDILDQALXXPGRIDRKMNF